(19) United States
(12) Patent Application Publication
(10) Pub. No.: US 2023/0083418 A1
(43) Pub. Date: Mar. 16, 2023

(54) MACHINE LEARNING SYSTEM FOR THE INTELLIGENT MONITORING AND DELIVERY OF PERSONALIZED HEALTH AND WELLBEING TOOLS

(71) Applicant: Microsoft Technology Licensing, LLC, Redmond, WA (US)

(72) Inventors: Daniel J. MCDUFF, Cambridge, MA (US); Javier HERNANDEZ RIVERA, Sommerville, MA (US); Mary P. CZERWINSKI, Kirkland, WA (US); Kael R. ROWAN, Box Elder, SD (US); Jin A. SUH, Seattle, WA (US); Gonzalo A. RAMOS, Kirkland, WA (US)

(73) Assignee: Microsoft Technology Licensing, LLC, Redmond, WA (US)

(21) Appl. No.: 17/474,347

(22) Filed: Sep. 14, 2021

(51) Int. Cl. G16H 20/70

(52) U.S. Cl. CPC G16H 20/70

(57) ABSTRACT

Various methods and apparatus relating to estimating and mitigating a stress level of a user are disclosed herein. Methods can include collecting potential stress indicator data from the user interacting with a computing device. The potential stress indicator data can include one or more of environmental data and contextual data associated with the user. Methods can include estimating the stress level of the user based on the potential stress indicator data. Methods can include performing an evaluation of whether to mitigate the stress level of the user via one or more stress mitigation interventions. Methods can include presenting the one or more stress mitigation interventions to the user via a graphical user interface (GUI) when the evaluation indicates that the stress level should be mitigated.

### BACKGROUND

Across nearly all industries, technological advances have led to increased workplace productivity. While most of technological advances are focused on how to help employees perform their jobs more efficiently, employee health is a significant factor that can also help increase productivity. For instance, employees in workplaces, across all industries, may experience stress-inducing activities, poor diet, long periods of sedentary activities, and unhealthy sleep behaviors. In addition, an increasing number of workers are participating in remote/hybrid work, which has blurred the lines between professional and personal lives and imposed additional demands and may lead to some unhealthy habits to occur at home. This may result in an increased strain on mental health and wellness and also lead loss of productivity overall.

It is with respect to these and other general considerations that embodiments have been described. Also, although relatively specific problems have been discussed, it should be understood that the embodiments should not be limited to solving the specific problems identified in the background.

### SUMMARY

Aspects of the present disclosure generally relate to the use of a computing devices, and more particularly, to apparatus and methods for perceiving a physical and emotional state using a computing device. Over the last decades, the information technology industry has seen a significant increase in health-related efforts, most significantly in the form of wearables to monitor user activity. These wearables, however, can be inaccessible to users for a variety of reasons (e.g., high cost), can experience high attrition rates (e.g., due to limited utility, poor aesthetics, high maintenance, social stigma), and can create costly noise in most of the sensed signals (e.g., heart rate). In addition, these wearables tend to ignore contextual information that may be useful in interpreting the perceived signal (e.g., elevated heart rates during exercise versus while watching a movie.) Aspects disclosed herein can advantageously achieve a rich contextual understanding of a user's activity as they navigate through a suite of contextually rich productivity tools on a computer. In addition, these achievements can be performed using standard sensors on most modern computers and has the potential to be augmented through additional devices (e.g., wearables, mobile devices, etc.).

In a first example, methods of mitigating a stress level of a user are disclosed herein. Such a method can include collecting potential stress indicator data from the user interacting with a computing device. The potential stress indicator data can include one or more of environmental data and contextual data associated with the user. The method can include estimating the stress level of the user based on the potential stress indicator data. The method can include performing an evaluation of whether to mitigate the stress level of the user via one or more stress mitigation interventions. The method can include presenting the one or more stress mitigation interventions to the user via a graphical user interface (GUI) when the evaluation indicates that the stress level should be mitigated. In examples, the environmental data can include data that is indicative of one or more of physiological activity and behavioral activity of the user. The contextual data can include data that is indicative of one or more of an amount of user interaction with the computing device and an amount of user activity away from the computing device.

Continuing with the first example, the method can utilize one or more computing devices comprising at least one input device with which to perceive the potential stress indicator data and a display with which to present the one or more stress mitigation interventions. In examples, the computing device is a personal computer, and the potential stress indicator data includes at least one of pointer activity, mouse activity, keyboard activity, and personal information manager activity.

Further toward the first example, the method can employ a machine learning (ML) stress mitigation architecture. The ML stress mitigation architecture can include a stress indicator model that is configured to identify actual stress indicator data in the collected potential stress indicator data. The ML stress mitigation architecture can include a stress estimator model that is configured to estimate the stress level of the user. In this regard, estimating the stress level of the user based on the potential stress indicator data can be a multi-operation process. For instance, this estimating operation can include identifying, via the stress indicator model, actual stress indicator data from the collected potential stress indicator data. The estimating operation can include aggregating, via the stress estimator model, the actual stress indicator data into an estimated stress level of the user.

In a second example, systems and methods for estimating a stress level of a user interacting with a computing device are disclosed herein. Such aspects can include selecting a baseline stress level of the user. Aspects disclosed herein can include collecting potential stress indicator data from the user interacting with the computing device. The potential stress indicator data can include one or more of environmental data and contextual data associated with the user. Aspects disclosed herein provide for estimating the stress level of the user based on the potential stress indicator data and returning the stress level to a graphical user interface (GUI) the computer device. In examples, estimating the stress level of the user based on the potential stress indicator data can be a multi-operation process. This multi-operation process can include processing the potential stress indicator data to identify actual stress indicator data. This multi-operation process can further include aggregating the actual stress indicator data to form the stress level of the user. The stress level of the user to can be compared to the baseline stress level of the user.

In a third example, graphical user interfaces (GUIs) for mitigating a stress level of a user are disclosed herein. Such a GUI can include a workspace with which to display applications running on a computing device. The GUI can be configured to receive an indication that mitigation of the stress level of the user is recommended. In examples, this indication can occur upon estimation of the stress level of the user based on potential stress indicator data from the user interacting with the system of computing devices associated with the user. The potential stress indicator data can include one or more of environmental data and contextual data associated with the user. The GUI can be configured to modify the workspace to include a wellness widget that is configured to provide a stress mitigating intervention to the user.

This Summary is provided to introduce a selection of concepts in a simplified form that are further described below in the Detailed Description. This summary is not intended to identify key features or essential features of the claimed subject matter, nor is it intended to be used to limit the scope of the claimed subject matter. Additional features and advantages of the present disclosure will become apparent to those skilled in the art upon consideration of the following detailed description of the illustrative embodiments exemplifying the disclosure as presently perceived.

### BRIEF DESCRIPTION OF THE DRAWINGS

Non-limiting and non-exhaustive examples are described with reference to the following Figures.

FIG. 1 is a schematic diagram of a system for estimating and mitigating stress levels of a user interacting with a computing device, according to principles of the present disclosure;

FIG. 2 is a flowchart of a method of estimating the stress level of a user interacting with a computing device, according to principles of the present disclosure;

FIG. 3 is a flowchart of a method of mitigating the stress level of a user interacting with a computing device, according to principles of the present disclosure;

FIG. 4A is a schematic diagram of a display with a GUI having a horizontally modified workspace, according to principles of the present disclosure;

FIG. 4B is a schematic diagram of a display with a GUI having a vertically modified workspace, according to principles of the present disclosure;

FIG. 4C is a schematic diagram of a display with a GUI having a modified workspace with a window, according to principles of the present disclosure; and

FIG. 4D is a schematic diagram of a display with a calendar application on GUI having a modified workspace, according to principles of the present disclosure.

FIG. 5 is a block diagram illustrating example physical components of a computing device with which aspects of the disclosure may be practiced.

FIGS. 6A and 6B are simplified block diagrams of a mobile computing device with which aspects of the present disclosure may be practiced.

FIG. 7 is a simplified block diagram of a distributed computing system in which aspects of the present disclosure may be practiced.

### DETAILED DESCRIPTION OF THE DRAWINGS

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Embodiments may be practiced as methods, systems or devices. Accordingly, embodiments may take the form of a hardware implementation, an entirely software implementation, or an implementation combining software and hardware aspects. The following detailed description is therefore not to be taken in a limiting sense, and the scope of the present disclosure is defined by the appended claims and their equivalents.

The pace of work in modern society has been quickly increasing over the last decade. While technical advancements in software have allowed employees to achieve more in less time, such advancements tend not to focus on helping individuals address behaviors that negatively affect their health, such as staying up late, consuming too much caffeine, scheduling back-to-back sedentary meetings, etc. Furthermore, the unique circumstances of work from home and remote/hybrid work have blurred the lines between professional and personal lives imposing unusual demands and potentially unhealthy habits to workers. This culture is not only detrimental to mental health and wellness but, in the long-run, may to lead to a loss of productivity and may lead to increased costs on society in general (e.g., resulting in increased medical and insurance costs).

Over the last decades, the information technology industry has seen a significant increase in health-related efforts, most significantly in the form of wearables to monitor user activity. These wearables, however, can be inaccessible to users for a variety of reasons (e.g., high cost), can experience high attrition rates (e.g., due to limited utility, poor aesthetics, high maintenance, social stigma), and can create costly noise in most of the sensed signals (e.g., heart rate). In addition, these wearables tend to ignore contextual information that may be useful in interpreting the perceived signal (e.g., elevated heart rates during exercise versus while watching a movie). Aspects disclosed herein can advantageously achieve a rich contextual understanding of a user's activity as they navigate through a suite of contextually rich productivity tools on a computer. In addition, these achievements can be performed using standard sensors on most modern computers and has the potential to be augmented through additional devices (e.g., wearables, mobile devices, etc.).

Aspects of the present disclosure provide a holistic solution which supports information workers to maximize their emotional resilience and well-being. Leveraging machine learning, among other features, aspects disclosed herein may be capable of passively sensing physiological and behavioral signals of the user (e.g., heart rate, respiration, typing dynamics) without the use of any wearable. Aspects disclosed herein capture relevant contextual information (e.g., calendar events, notifications) that is used along with behavioral signals to assess the stress levels of the user, the originating sources of stress, and the best timing to help users manage their stress. When help is needed, aspects disclosed herein are operable to display personalized guidance and suggestions to provide immediate stress relief and teach long-term coping skills. For instance, the disclosed aspects may recommend performing interactive breathing exercises, offloading the user's mind or release negative thoughts with a game, and/or other stress relieving activities. Furthermore, aspects of the present disclosure also provides a reflective dashboard that allows users to introspect into the most likely sources of workplace stress as well as potential guidelines to help alleviate the stresses. Exemplary systems and methods may further recommend re-scheduling cognitive-demanding tasks at certain times of the day based on circadian rhythms, scheduling relaxing interventions after stressful meetings, or point out at useful resources that may help better meet certain demands.

In order to preserve user privacy, the user is provided with the ability to opt-in to the aspects disclosed herein. Only when the user enables the opt-in mode will the aspects disclosed herein be able to learn the user's computer habits and workplace stressors and to make automatic subtle changes on the user's system environment to dynamically support a positive, productive state for the user. Some of these changes may include playing background audio signals to block distractions and remain focused, setting nature backgrounds on the desktop to help set the proper tone, or blocking certain types of notifications when stress is high.

Existing solutions usually involve the development of wearables to help monitor users. However, wearables can be inaccessible due to high cost and experience high attrition rates due to several human factors (e.g., limited utility, poor aesthetics, high maintenance, social stigma). In addition, body motion usually creates a lot of noise in most of the sensed signals, such as heart rate, which severely limits their potential value. Finally, these devices tend to ignore contextual information, which plays a critical role when interpreting the signals. For instance, increased heart rate at the gym and increased heart rate at the movies are interpreted in the same way by most wearables. Aspects of the present disclosure provide benefits over existing solutions in that the systems and methods disclosed herein do not require the use of wearable technology (although wearable technology can be incorporated into the systems and methods disclosed herein). Furthermore, the aspects disclosed herein are capable of processing information from sources other than a wearable, providing, among other benefits, the ability to determine a cause or source of user stress, a correlation of an activity or behavior to stress, etc.

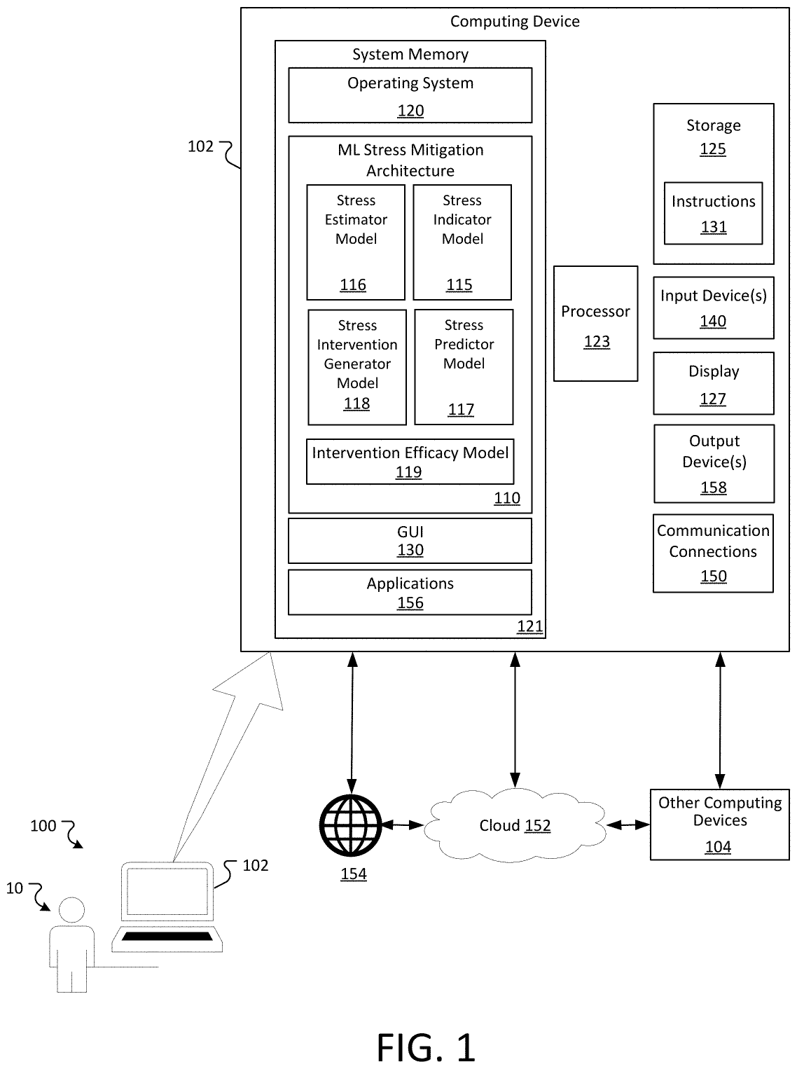

FIG. 1 shows a schematic diagram of a system 100 employing principles of the present disclosure. As backdrop, described herein is a holistic system 100 with services that promote the wellbeing of a user 10 in the workplace. As noted above, in consideration of user privacy, the user 10 can opt-in to use of the system 100. For instance, the system 100 can be implemented on a computing device 102 such as a personal computer and networked with other devices 104 (e.g., wearables, mobile devices, etc.). Leveraging the latest advancements in human-centered AI, the system 100 can passively sense environmental signals (e.g., physiological and behavioral signals) associated with the user 10 without requiring the use of any wearable. For instance, the system 100 can use machine learning (ML) stress mitigation architecture 110, which is discussed in further detail below. In general, the ML stress mitigation architecture 110 can include a stress indicator model 115, a stress estimator model 116, a stress predictor model 117, a stress intervention generator model 118, and an intervention efficacy model 119. While the ML stress mitigation model is described as comprising a specific set of models, one of skill in the art will appreciate that more or fewer models may be employed without departing from the scope of this disclosure. Similarly, the functionality of the different models may be combined or split without departing form the scope of this disclosure. The system 100 can gather relevant physiological data (e.g., heart rate, respiration, mouse activity, typing dynamics, etc.) and contextual data (e.g., emails, calendar events, in-use applications, notifications, etc.) to assess the stress levels of the user 10, determine its originating sources, and decipher the best timing and interventions to help users manage it. As will be appreciated, a multiple-device system 100 employing several other devices 104 (e.g., other computing devices 104) associated with the user 10 (e.g., through account authentication) can provide more environmental and contextual data to increase the efficacy of the system 100. In addition, single-tenant or multi-tenant platforms and local or cloud-based systems 152 can be employed to meet the needs of a particular implementation of the system 100 and to meet certain performance criteria.

When appropriate, the system 100 can display 127 personalized guidance and suggestions to mitigate unhealthy amounts of stress and teach long-term coping skills. For instance, the system 100 may recommend performing interactive breathing exercises, offloading cognitive loads, or playing games to release negative thoughts. In certain instances, the system 100 can provide one or more dashboards that allow users to introspect into the most likely sources of workplace stress as well as potential guidelines to help alleviate them. In certain instances, the system 100 may recommend re-scheduling tasks with high-cognitive demands to certain times of the day (e.g., based on circadian rhythms), scheduling relaxing interventions after stressful meetings, or using helpful resources to better meet certain demands.

Of note, the system 100 can include an opt-in mode that learns a user's habits. For instance, the system 100 can learn computer habits and workplace stressors to in turn make automatic subtle changes on the user's system environment to dynamically support a positive, productive state. Some of these changes may include playing background audio signals to block distractions and remain focused, setting nature backgrounds on the desktop to help set the proper tone, or blocking certain types of notifications when stress is high. If the user does not opt in, the system 100 will not collect information about the user.

As illustrated in FIG. 1, the system 100 includes a computing device 102 in the form of a personal computer with numerous peripheral devices. Computing devices may include any suitable device, such as a desktop computer, laptop, tablet, mobile computing device (e.g., smartphone or smartwatch), or other suitable device with an operating system 120. As will be appreciated, the computing device 102 can include a memory 121, a processor 123, a storage 125, and a display 127 on which a graphical user interface 130 (GUI 130) is used to provide input to the computing device 102 to perform certain user-desired functions on the operating system 120. The processor 123 can employ certain logic directed by the user 10 or the system 100, and the storage 125 can be used to store, among other things, instructions 131 for directing the processor 123 to perform certain functions (e.g., via non-transitory computer-readable mediums). As will be understood, the computing device 102 can have the operating system 120 associated with or otherwise operable to display the GUI 130 such that the GUI 130 is presented on the display 127 to allow a user 10 to interact with the computing device 102. These interactions are performed using one or more peripheral input devices 140 (e.g., a touchscreen, a computer mouse, a keyboard, a webcam, a computer pen, etc.). Communication connections 150 in the form of a modem or network interface controller can connect the computing device 102 to other network devices, cloud-based systems 152, and/or the internet 154. Running on the computing device 102 can be one or more applications 156, including productivity applications such as word processing, personal information manager, and other local applications as well as web-based applications. Peripheral output devices 158 such as projectors and speakers can also be included in the computing device.

Data streams from these and more applications running on the computing device 102 include environmental and contextual data about the user 10 and their associated activity. Thus, when properly parsed, these data streams can provide indicators of a user's activity (e.g., throughout the workday or workweek) and associated stress levels. Even more, machine learning principles employed through models can learn from these data streams and train the models to provide to the user 10 personalized recommendations for mitigated their stress levels. More details about these principles of the present disclosure are provided below.

### Stress Level Estimation

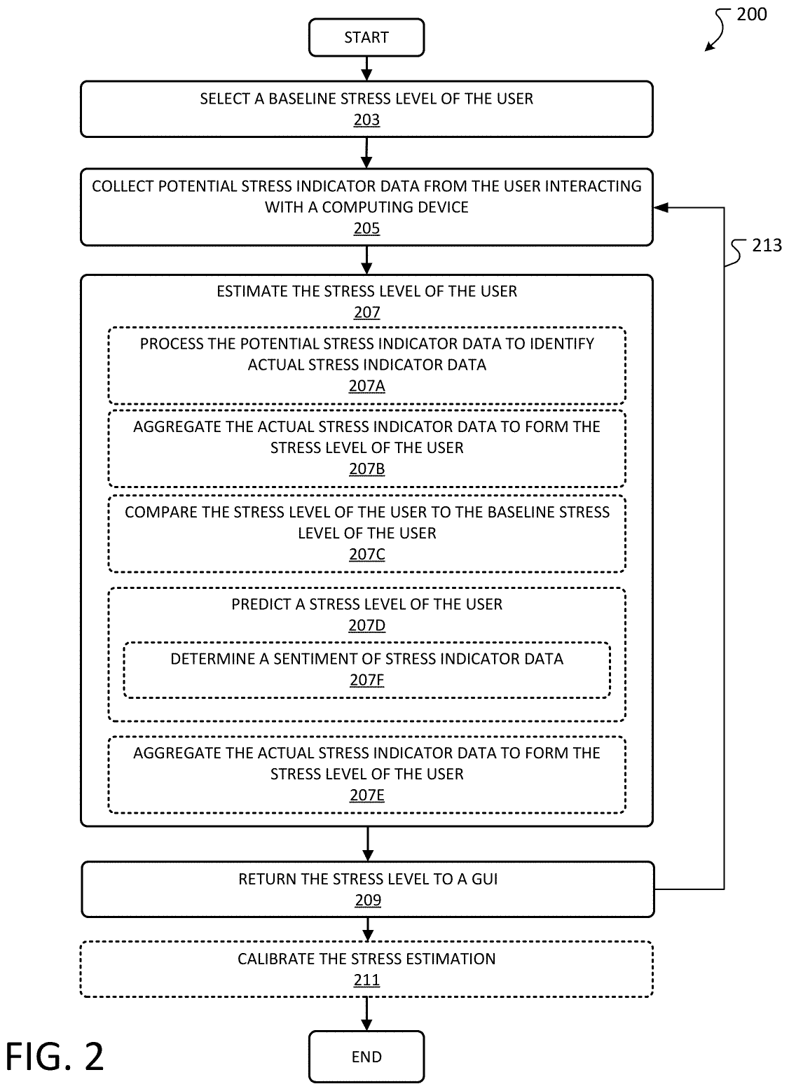

Disclosed herein are methods of estimating a stress level of a user interacting with a computing device as shown in the flowchart FIG. 2. The method 200 can include selecting a baseline stress level of the user at operation 203. The method 200 can include collecting potential stress indicator data from the user interacting with the computing device at operation 205. The potential stress indicator data can include one or more of environmental data and contextual data associated with the user. The method 200 can include estimating the stress level of the user based on the potential stress indicator data at operation 207. The method 200 can include returning the stress level to a graphical user interface (GUI) of the computer device at operation 209. As a summary to further discussion provided below, the GUI can include a workspace with which to display applications running on the computing device. The GUI can be configured to perform at least one of modifying the workspace to include a wellness widget, providing the one or more stress mitigating interventions to the user, and displaying an estimated stress of the user over time.

In examples, the method 200 can be performed via ML stress mitigation architecture 110. The ML stress mitigation architecture can include one or more ML models with which to perform various operations of the method 200. In examples, there may be an ML model for individual potential stressors, interventions, or both. Alternatively, or in addition, there may be an ML model that generally monitors multiple stressors, intervention, or both. By way of example, the ML stress mitigation architecture can include a stress indicator model that is configured to identify actual stress indicator data in the collected potential stress indicator data. The ML stress mitigation architecture can include a stress estimator model that is configured to estimate the stress level of the user.

In this regard, estimating the stress level of the user based on the potential stress indicator data can be a multi-operation process. This multi-operation process can include processing the potential stress indicator data at operation 207A to identify actual stress indicator data. For instance, this estimating operation can include identifying actual stress indicator data from the collected potential stress indicator data via the stress indicator model. This multi-operation process can include aggregating the actual stress indicator data at operation 207B to form the stress level of the user. For instance, this estimating operation can include aggregating the actual stress indicator data into an estimated stress level of the user via the stress estimator model. The method 200 can include comparing the stress level of the user to the baseline stress level of the user at operation 207C.

While discussed in terms of current or past stress levels above, this disclosure also contemplates predictive stress estimation. In examples, the ML stress mitigation architecture can include a stress predictor model that is configured to predict a future stress level of the user based on the potential stress indicator. In this regard, estimating the stress level of the user based on the potential stress indicator data at operation 207 can include predictive estimates. For instance, this operation 207 can include predicting the future stress level of the user at operation 207D via the stress predictor model. For instance, this operation 207 can include aggregating the future stress level of the user into the estimated stress level of the user via the stress estimator model 207E. In examples, estimating the stress level of the user based on the potential stress indicator data can include at least one of determining a current stress level of the user and predicting a future stress level of the user.

In examples, predicting the stress level of the user at operation 207D can include determining a sentiment of stress indicator data at operation 207F. This operation 207F can be performed for current or future stress level. In examples, determining a sentiment of stress indicator data at operation 207F can be from a personal information manager in communication with the system of computing devices. For instance, the system can parse data associated with emails, calendar invitations, and other textual instances on the computing device to determine whether they escalated or deescalated the user's stress level. This estimation can then be compared to the user's baseline stress level to determine whether a stress mitigation intervention is appropriate as further discussed below.

In examples, estimating the stress level of the user based on the potential stress indicator data is performed at operation 207 via a stress estimator model. In this regard, the method 200 can include calibrating the stress estimator model at operation 211 via one or more user prompts that are configured to provide indication of an actual stress level of the user. For instance, the method 200 can include providing, periodically, the user with a survey that is indicative of at least one of an efficacy of the one or more stress mitigation interventions, a mood of the user, and demographic information about the user. This information, in turn, can be used to inform the ML stress mitigation architecture as it operates to estimate the stress level of the user (as illustrated via feedback loop 213).

Calibrating the stress estimator model at operation 211 can lead to improved system performance. For example, in examples of the method 200, calibration can occur via one or more user prompts that are configured to provide indication of an actual stress level of the user. The prompts can be in the form of surveys or user textual or audio dialogues with the computing device, for example. In some such examples, the evaluation of whether to mitigate, via one or more stress mitigation interventions, the stress level of the user is performed over an evaluation window that indicates a change in stress over time.

Implementations of the method 200 can be performed using a computing device as previously discussed. For instance, the computing device can include at least one input device with which to perceive the potential stress indicator data and a display with which to present the one or more stress mitigation interventions. In examples, the computing device is a personal computer, and the potential stress indicator data includes at least one of pointer activity, mouse activity, keyboard activity, and personal information manager activity. In examples, the environmental data can include data that is indicative of one or more of physiological activity and behavioral activity of the user. The contextual data can include data that is indicative of one or more of an amount of user interaction with the computing device and an amount of user activity away from the computing device.

Perhaps best understood in the context of a non-limiting example, using principles of the present disclosure, the system disclosed herein can estimate stress levels of users in the workplace. In this regard, each user in the workplace can perform their duties primarily on a personal computer with connected peripherals and personal devices. As the user goes about their workday, numerous keystrokes, mouse movements, and facial expressions can be captured by the system. As well, the system can passively determine sentiments associated with certain applications, such as email and calendar applications, that are running on the personal computer. Throughout the day, at designated intervals or in comparison to previous days or weeks, the system can estimate the stress level of the user by accessing data streams from the numerous inputs as environmental indicators of stress as well as data streams from the applications determined sentiments. When ML models are employed, feedback loops (e.g., using user responses to surveys) can calibrate the system to better estimate the stress level of the user. As further discussed below, in response to, on user demand, and/or proactively, the system can generate one or more stress mitigation interventions to mitigate the stress level. Continued discussion of this example with other principles disclosed herein is included below.

### Stress Mitigation Intervention Generation

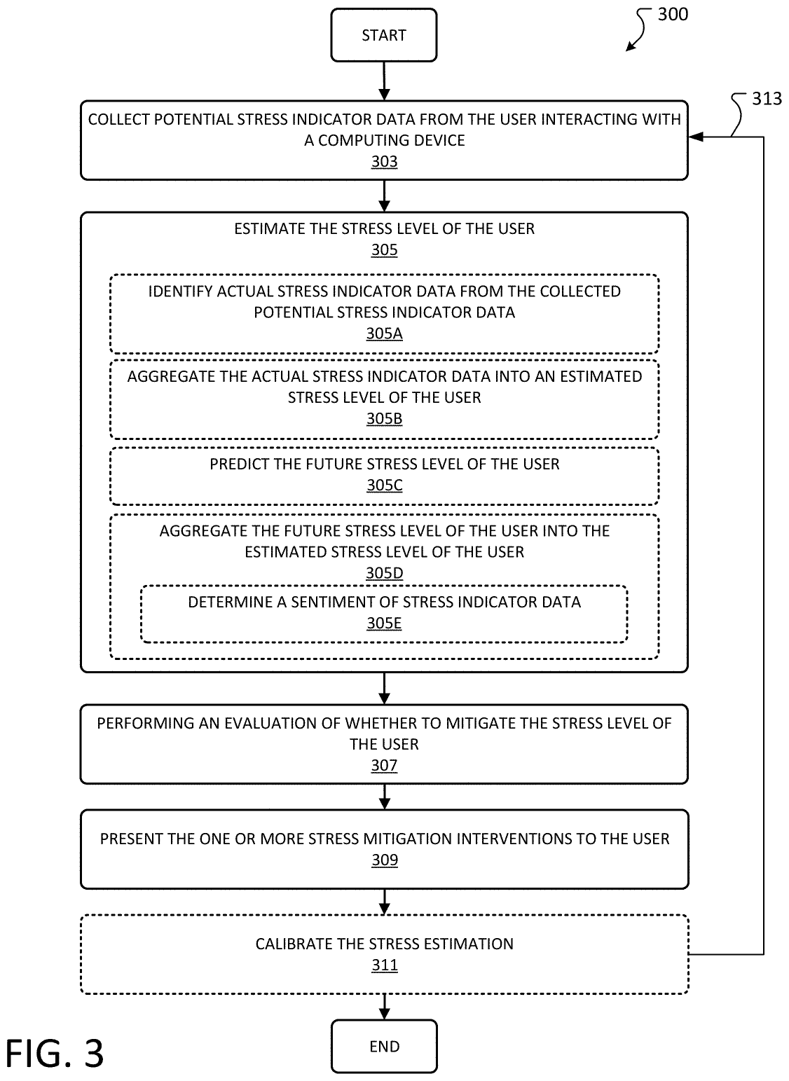

Methods of mitigating a stress level of a user are disclosed herein as shown in the flowchart FIG. 3. The method 300 can include collecting potential stress indicator data at operation 303 from the user interacting with a computing device. The potential stress indicator data can include one or more of environmental data and contextual data associated with the user. The method 300 can include estimating the stress level of the user at operation 305 based on the potential stress indicator data. The method 300 can include performing an evaluation of whether to mitigate the stress level of the user at operation 307 via one or more stress mitigation interventions. The method 300 can include presenting the one or more stress mitigation interventions to the user at operation 309 via a graphical user interface (GUI) when the evaluation indicates that the stress level should be mitigated.

As alluded to above, estimating the stress level of the user based on the potential stress indicator data can be a multi-operation process. This multi-operation process can include processing the potential stress indicator data at operation 305A to identify actual stress indicator data. For instance, this estimating operation can include identifying actual stress indicator data from the collected potential stress indicator data via the stress indicator model. This multi-operation process can include aggregating the actual stress indicator data at operation 305B to form the stress level of the user. For instance, this estimating operation can include aggregating the actual stress indicator data into an estimated stress level of the user via the stress estimator model.

While discussed in terms of current or past stress levels above, this disclosure also contemplates predictive stress estimation. In examples, the ML stress mitigation architecture can include a stress predictor model that is configured to predict a future stress level of the user based on the potential stress indicator. In this regard, estimating the stress level of the user based on the potential stress indicator data at operation 305 can include predictive estimates. For instance, this operation 305 can include predicting the future stress level of the user at operation 305C via the stress predictor model. For instance, this operation 305 can include aggregating the future stress level of the user into the estimated stress level of the user via the stress estimator model 305D. In examples, estimating the stress level of the user based on the potential stress indicator data can include at least one of determining a current stress level of the user and predicting a future stress level of the user.

In examples, predicting the stress level of the user at operation 305C can include determining a sentiment of stress indicator data at operation 305E. This operation 305E can be performed for current or future stress level. In examples, determining a sentiment of stress indicator data at operation 305E can be from a personal information manager in communication with the system of computing devices. For instance, the system can parse data associated with emails, calendar invitations, and other textual instances on the computing device to determine whether they escalated or deescalated the user's stress level. This estimation can then be compared to the user's baseline stress level to determine whether a stress mitigation intervention is appropriate as further discussed below.

Stress mitigation interventions can vary in degree corresponding to at least one of the stress level and an efficacy of the one or more stress mitigation interventions. Regarding their type, the one or more stress mitigation interventions can include distraction interventions, relaxation interventions, and thought modifying interventions. Further, stress mitigation interventions can be active (e.g., those that require active attention of the user so s/he needs to pause work such as meditation or talking to a friend) or passive (e.g., those that do not require active attention of the user so s/he can continue to work such as listening to music on the background or changing the temperature). Regarding their complexity, the one or more stress mitigation interventions can increase in the level of difficulty, duration, or degree of their intended effect for example. Efficacy of the one or more stress mitigation interventions can be measured in a variety of ways, including based on feedback from the user, feedback derived from collected data streams, and/or a completion percentage. As noted above, in examples, the method 300 can include providing, periodically, the user with a survey that is indicative of at least one of an efficacy of the one or more stress mitigation interventions, a mood of the user, and demographic information about the user. This information, in turn, can be used to inform the ML stress mitigation architecture as it operates to mitigate the stress of the user.

An ML stress mitigation architecture (such as the one discussed above or an independent one) can include a stress intervention generator model that is configured to vary at least one of a complexity and a type of the one or more stress mitigation interventions corresponding to at least one of the stress level and the efficacy of the one or more stress mitigation interventions. As with other ML models disclosed herein, the stress intervention generator model can be personalized to the user's stress level and can be trained to make appropriate variations when appropriate. In examples, the ML stress mitigation architecture can include an intervention efficacy model that is configured to determine one or more of an efficacy of the intervention on the stress level of the user and whether the user completes the intervention.

Stress mitigation intervention generation can include user interaction. While in some instances the system can trigger this generation, some instances may be facilitated using user interaction. For instance, the system may decide to immediately trigger a stress mitigation intervention and, in other instances, the system may decide to suggest or place an intervention on the user's calendar for a future time. At certain points, the system may provide the user with several options of interventions (e.g., active or passive) to choose from. On the other hand, the user may trigger interventions on demand at any time. The system may use these instanced of on-demand intervention generation to better inform its triggering of stress mitigation intervention generation.

As was the case in previously discussed methods, estimating the stress level of the user based on the potential stress indicator data is performed at operation 305 via a stress estimator model. In this regard, the method 300 can include calibrating the stress estimator model at operation 311 via one or more user prompts that are configured to provide indication of the efficacy of an intervention on an actual stress level of the user. For instance, as alluded to previously, the method 300 can include periodically providing the user with a survey that is indicative of at least one of an efficacy of the one or more stress mitigation interventions, a mood of the user, and demographic information about the user. This information, in turn, can be used to inform the ML stress mitigation architecture as it operates to determine the efficacy of an intervention on the stress level of the user (as illustrated via feedback loop 313).

Calibrating the stress estimator model at operation 311 can lead to improved system performance. For example, in examples of the method 300, calibration can occur via one or more user prompts that are configured to provide indication of the efficacy of an intervention on an actual stress level of the user. The prompts can be in the form of surveys or user textual or audio dialogues with the computing device, for example. In some such examples, the evaluation of whether to mitigate, via one or more stress mitigation interventions, the stress level of the user is performed over an evaluation window that indicates a change in stress over time.

Continuing with the non-limiting example discussed above, in addition or in alternative thereto, the system disclosed herein can mitigate stress levels of users in the workplace. After receiving indication of user stress levels that need to be mitigated, the system can intervene with one or more stress mitigation interventions. For instance, when user stress levels are elevated, the system can intervene with a personalized stress mitigation intervention in the form of physical activities, activities at the computer, activities away from the computer or other appropriate interventions. In certain instances, the system may determine (e.g., based on sentiments derived from a calendar application running on the computer, that certain scheduled activities (e.g., lunch break, walk, etc.) operate as a stress mitigation intervention and account for these interventions in how and when it provides (presently or in the future) additional interventions. After an intervention is completed, the system may prompt the user to answer survey questions that indicate the efficacy of the intervention and/or monitor data streams to make similar determinations. This information can be used to inform the ML stress mitigation architecture on what interventions are effective for which purposes. These interactions, and others discussed herein, can be facilitated via a GUI running on the computing device.

### Stress Estimation and Mitigation GUI

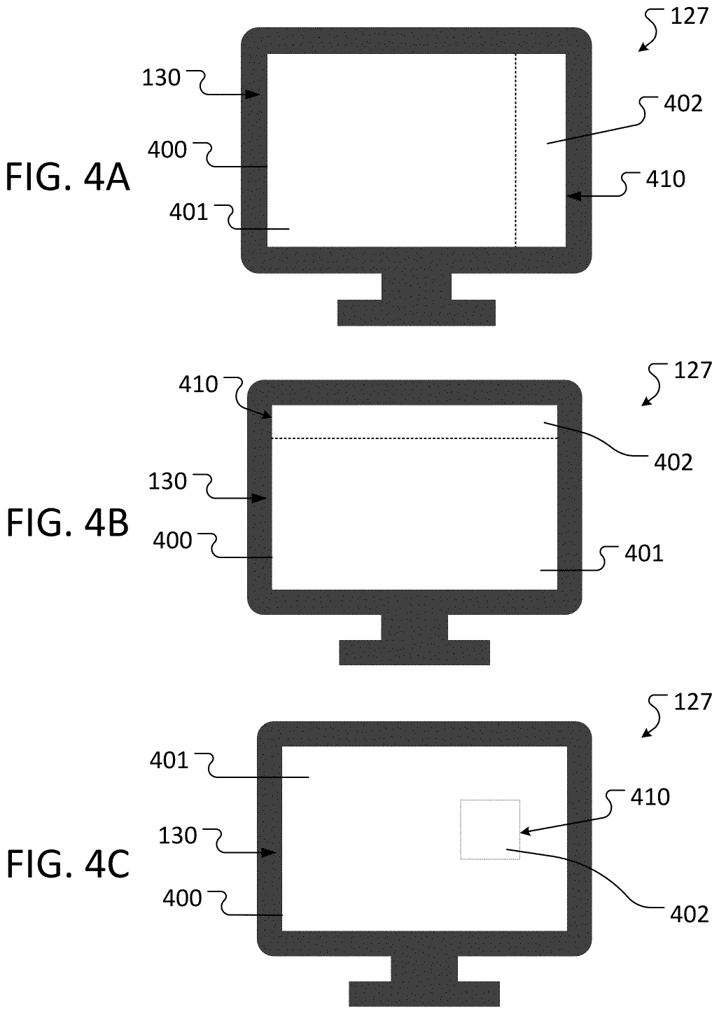

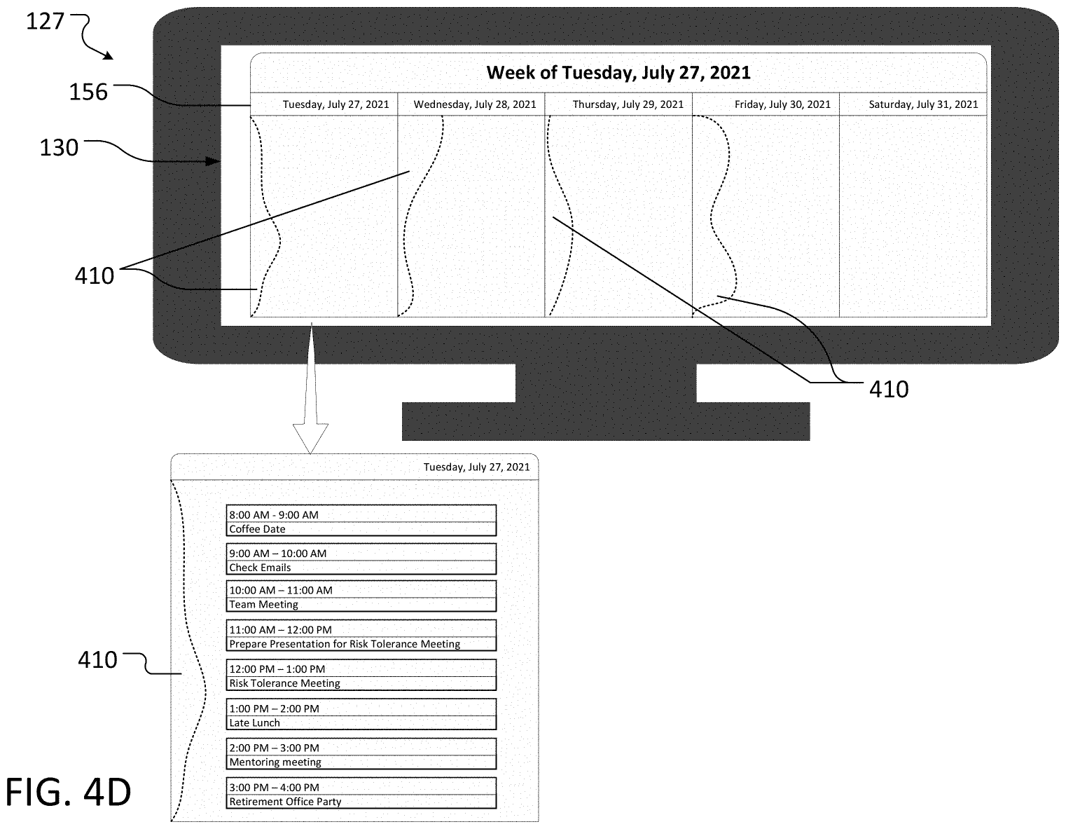

Disclosed herein are graphical user interfaces (GUIs) for mitigating a stress level of a user as shown in FIGS. 4A-4D, which show various features of a display 127. The GUI 130 has a workspace 400 that can be modified into multiple workspaces (e.g., a first workspace 401 and a second workspace 402). FIG. 4A shows the GUI 130 with a wellness widget 410 in the form of a sidebar. FIG. 4B shows the GUI 130 with a wellness widget 410 in the form of a top bar. FIG. 4C shows the GUI 130 with a wellness widget 410 in the form of a window. FIG. 4D shows a wellness widget 410 in the form of a graph that is integrated into an application to be presented on the GUI 130. The wellness widget 410 can be defined as any application, process, or manipulation (e.g., of the GUI 130 or processes) that is designed to mitigate the stress level of a user. As discussed below, the wellness widget 410 can take a variety of forms, such as a portion of a workspace 400 on the GUI 130 or in a display 127 within an application displayed on the GUI 130.

As shown in FIGS. 4A-4D, the GUI 130 can include a workspace 400 with which to display applications 156 running on a computing device. The GUI 130 can be configured to receive an indication that mitigation of the stress level of the user is recommended. In examples, this indication can occur upon estimation of the stress level of the user based on potential stress indicator data from the user interacting with the system of computing devices associated with the user. The potential stress indicator data can include one or more of environmental data and contextual data associated with the user. Stress estimation and/or mitigation for the GUI 130 can be performed using any of the previously discussed methods, included the method 200 and the method 300.

The GUI 130 can be configured to modify the workspace 400 to include a wellness widget 410 that is configured to provide a stress mitigating intervention to the user. In examples, modifying the workspace 400 to include the wellness widget 410 that is configured to provide the stress mitigating intervention to the user can include modifying the workspace 400 to include a first workspace 401 for displaying the applications 156 and a second workspace 402 for displaying the wellness widget 410. In examples, the first and second workspaces can be positioned side by side on the display 127. The first workspace 401 can be larger than the second workspace 402. In this regard, the wellness widget 410 can be defined as a sidebar on the workspace 400. This sidebar can display certain live backgrounds or images that positively affect the user's stress level. For instance, the sidebar can show a tranquility live background or one or more positive images (together or in succession). Of course, the “side bar” can be in the form of an interactive tool bar or display across (e.g., at the top or bottom) of the display or in a separate (e.g., floating or incorporated) window without departing from the scope of this disclosure.

With particular reference to FIG. 4D, the wellness widget 410 can be incorporated into an application that is displayed on the GUI 130. For instance, the wellness widget 410 can be in the form of code that generates certain audio sounds (e.g., white noise, waterfalls, streams, etc.). In certain instances, the wellness widget 410 may be in the form of a dashboard presented to the user to provide substantive feedback to the user for introspect. In other instances, the wellness widget 410 may be in the form of a graph incorporated into applications 156 displayed on the GUI 130. For instance, the graph can be a graph (e.g., a bar graph, scatter plot, line graph, or the like) that is laid across the user's calendar display to provide an indication of the stress level of the user over time. The graph can show relative level of stress across an evaluation window (e.g., minutes, hours, days, weeks, months, etc.). In this regard, the system can passively generate stress estimations at certain time intervals throughout the day for use in these and other aspects of the present disclosure.

Continuing with the non-limiting example discussed above, in addition or in alternative thereto, the system disclosed herein can incorporate a GUI 130 into estimating and mitigating stress levels of users in the workplace. Estimated stress levels can be displayed on personal information manager applications 156 (e.g., email and/or calendar applications) running on the computing device so that the user is provided ample opportunity to interact with their stress mitigation and to introspect about their stress levels. In addition, after stress levels have been estimated, the GUI 130 can be used to interact with the user to perform the stress mitigation interventions, gather feedback, and/or decipher additional activity by the user. The system can determine the sentiment of calendar events and modify the estimated stress accordingly. As shown here, “Risk Tolerance Meeting” is shown to increase the estimated stress from the beginning of the day and “Late Lunch” is shown as mitigating the estimated stress after the “Risk Tolerance Meeting.”

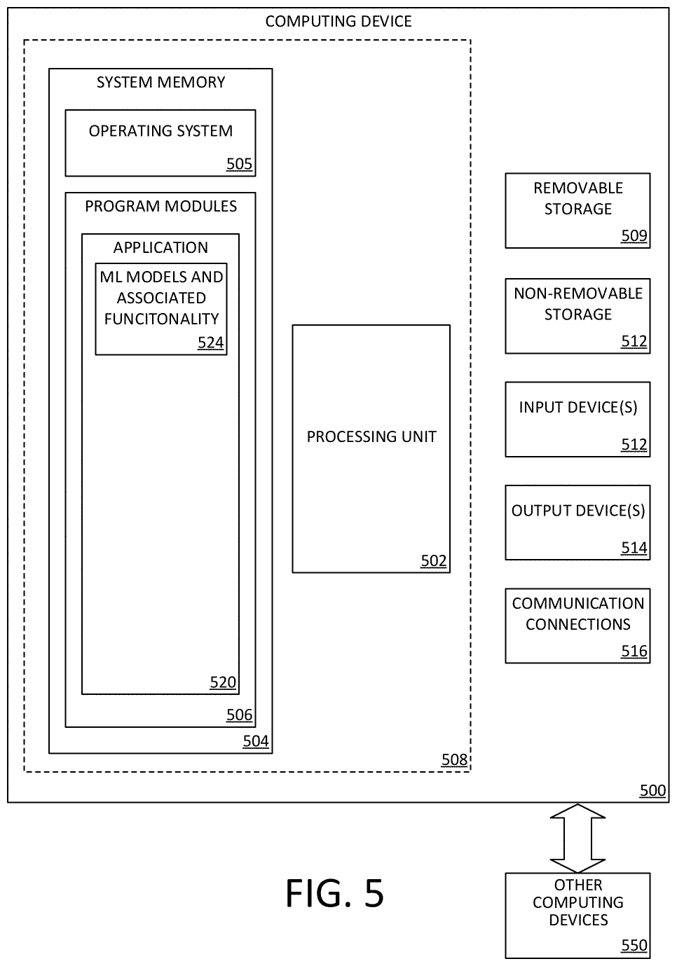

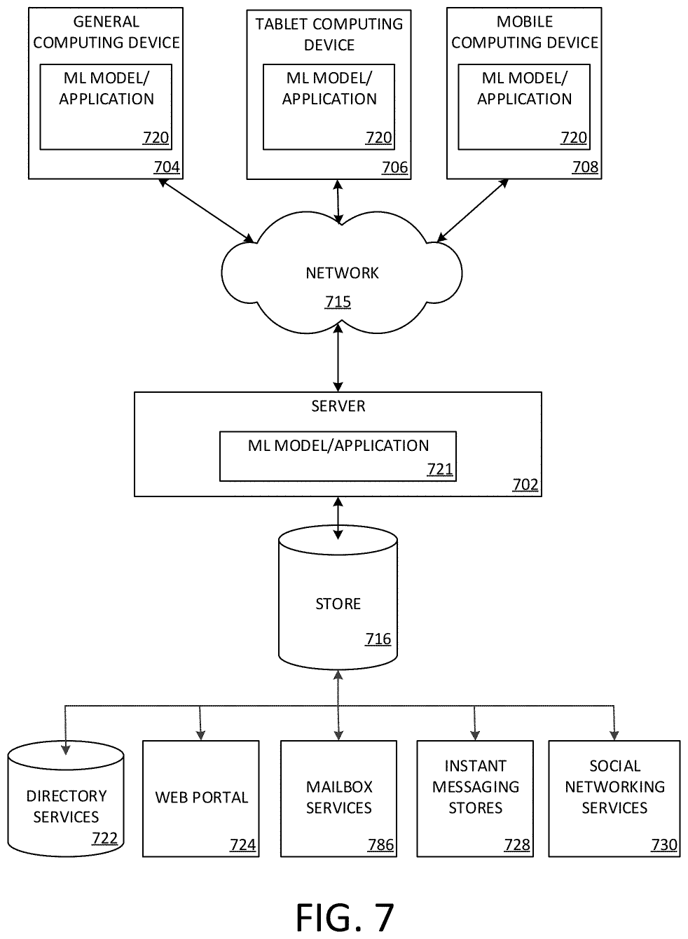

FIGS. 5-7 and the associated descriptions provide a discussion of a variety of operating environments in which aspects of the disclosure may be practiced. However, the devices and systems illustrated and discussed with respect to FIGS. 7-10 are for purposes of example and illustration and are not limiting of a vast number of computing device configurations that may be utilized for practicing aspects of the disclosure, described herein.

FIG. 5 is a block diagram illustrating physical components (e.g., hardware) of a computing device 500 with which aspects of the disclosure may be practiced. The computing device components described below may be suitable for the computing devices described above. In a basic configuration, the computing device 500 may include at least one processing unit 502 and a system memory 504. Depending on the configuration and type of computing device, the system memory 504 may comprise, but is not limited to, volatile storage (e.g., random access memory), non-volatile storage (e.g., read-only memory), flash memory, or any combination of such memories.

The system memory 504 may include an operating system 505 and one or more program modules 506 suitable for running software application 520, such as one or more components supported by the systems described herein. As examples, system memory 504 may store ML Models and or applications 524 performing functionality disclosed herein. The operating system 505, for example, may be suitable for controlling the operation of the computing device 500.

Furthermore, aspects of the disclosure may be practiced in conjunction with a graphics library, other operating systems, or any other application program and is not limited to any particular application or system. This basic configuration is illustrated in FIG. 5 by those components within a dashed line 508. The computing device 500 may have additional features or functionality. For example, the computing device 500 may also include additional data storage devices (removable and/or non-removable) such as, for example, magnetic disks, optical disks, or tape. Such additional storage is illustrated in FIG. 5 by a removable storage device 509 and a non-removable storage device 510.

As stated above, a number of program modules and data files may be stored in the system memory 504. While executing on the processing unit 502, the program modules 506 (e.g., application 520) may perform processes including, but not limited to, the aspects, as described herein. Other program modules that may be used in accordance with aspects of the present disclosure may include electronic mail and contacts applications, word processing applications, spreadsheet applications, database applications, slide presentation applications, drawing or computer-aided application programs, etc.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 5 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 500 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computing device 500 may also have one or more input device(s) 512 such as a keyboard, a mouse, a pen, a sound or voice input device, a touch or swipe input device, etc. The output device(s) 514 such as a display, speakers, a printer, etc. may also be included. The aforementioned devices are examples and others may be used. The computing device 500 may include one or more communication connections 516 allowing communications with other computing devices 550. Examples of suitable communication connections 516 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 504, the removable storage device 509, and the non-removable storage device 510 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 500. Any such computer storage media may be part of the computing device 500. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

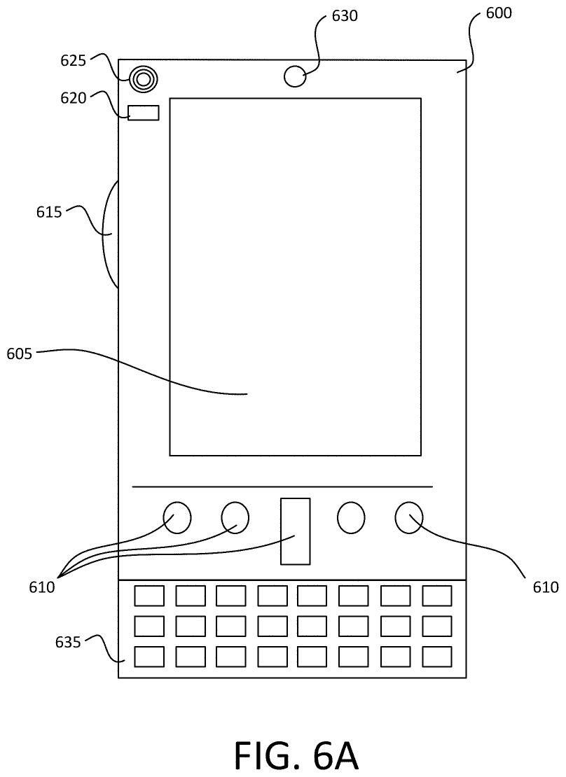

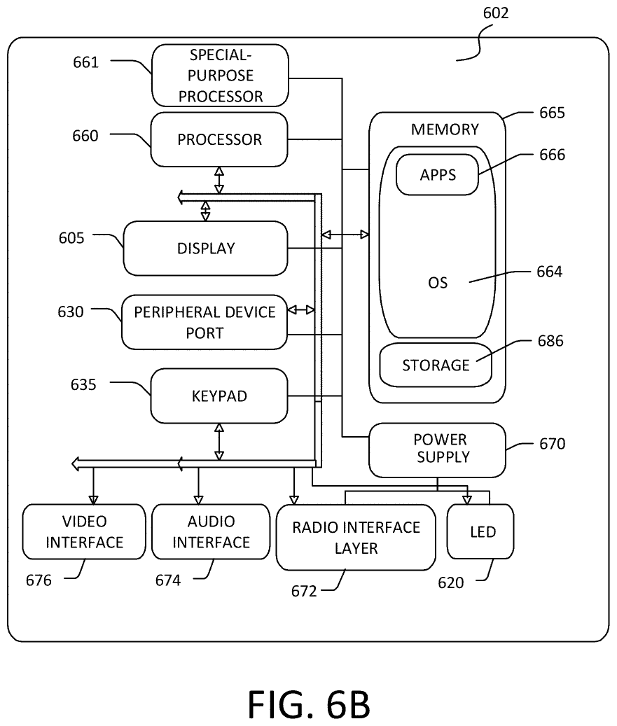

FIGS. 6A and 6B illustrate a mobile computing device 600, for example, a mobile telephone, a smart phone, wearable computer (such as a smart watch), a tablet computer, a laptop computer, and the like, with which embodiments of the disclosure may be practiced. In some aspects, the client may be a mobile computing device. With reference to FIG. 6A, one aspect of a mobile computing device 600 for implementing the aspects is illustrated. In a basic configuration, elements. The mobile computing device 600 typically includes a display 605 and one or more input buttons 610 that allow the user to enter information into the mobile computing device 600. The display 605 of the mobile computing device 600 may also function as an input device (e.g., a touch screen display).

If included, an optional side input element 615 allows further user input. The side input element 615 may be a rotary switch, a button, or any other type of manual input element. In alternative aspects, mobile computing device 600 may incorporate more or less input elements. For example, the display 605 may not be a touch screen in some embodiments.

In yet another alternative embodiment, the mobile computing device 600 is a portable phone system, such as a cellular phone. The mobile computing device 600 may also include an optional keypad 635. Optional keypad 635 may be a physical keypad or a “soft” keypad generated on the touch screen display.

In various embodiments, the output elements include the display 605 for showing a graphical user interface (GUI), a visual indicator 620 (e.g., a light emitting diode), and/or an audio transducer 625 (e.g., a speaker). In some aspects, the mobile computing device 600 incorporates a vibration transducer for providing the user with tactile feedback. In yet another aspect, the mobile computing device 600 incorporates input and/or output ports, such as an audio input (e.g., a microphone jack), an audio output (e.g., a headphone jack), and a video output (e.g., a HDMI port) for sending signals to or receiving signals from an external device.

FIG. 6B is a block diagram illustrating the architecture of one aspect of a mobile computing device. That is, the mobile computing device 600 can incorporate a system (e.g., an architecture) 602 to implement some aspects. In one embodiment, the system 602 is implemented as a “smart phone” capable of running one or more applications (e.g., browser, e-mail, calendaring, contact managers, messaging clients, games, and media clients/players). In some aspects, the system 602 is integrated as a computing device, such as an integrated personal digital assistant (PDA) and wireless phone.

One or more application programs 666 may be loaded into the memory 662 and run on or in association with the operating system 664. Examples of the application programs include phone dialer programs, e-mail programs, personal information management (PIM) programs, word processing programs, spreadsheet programs, Internet browser programs, messaging programs, and so forth. The system 602 also includes a non-volatile storage area 668 within the memory 662. The non-volatile storage area 668 may be used to store persistent information that should not be lost if the system 602 is powered down. The application programs 666 may use and store information in the non-volatile storage area 668, such as e-mail or other messages used by an e-mail application, and the like. A synchronization application (not shown) also resides on the system 602 and is programmed to interact with a corresponding synchronization application resident on a host computer to keep the information stored in the non-volatile storage area 668 synchronized with corresponding information stored at the host computer. As should be appreciated, other applications may be loaded into the memory 662 and run on the mobile computing device 600 described herein (e.g., search engine, extractor module, relevancy ranking module, answer scoring module, etc.).

The system 602 has a power supply 670, which may be implemented as one or more batteries. The power supply 670 might further include an external power source, such as an AC adapter or a powered docking cradle that supplements or recharges the batteries.

The system 602 may also include a radio interface layer 672 that performs the function of transmitting and receiving radio frequency communications. The radio interface layer 672 facilitates wireless connectivity between the system 602 and the “outside world,” via a communications carrier or service provider. Transmissions to and from the radio interface layer 672 are conducted under control of the operating system 664. In other words, communications received by the radio interface layer 672 may be disseminated to the application programs 666 via the operating system 664, and vice versa.

The visual indicator 620 may be used to provide visual notifications, and/or an audio interface 674 may be used for producing audible notifications via the audio transducer 625. In the illustrated embodiment, the visual indicator 620 is a light emitting diode (LED) and the audio transducer 625 is a speaker. These devices may be directly coupled to the power supply 670 so that when activated, they remain on for a duration dictated by the notification mechanism even though the processor 660 and other components might shut down for conserving battery power. The LED may be programmed to remain on indefinitely until the user takes action to indicate the powered-on status of the device. The audio interface 674 is used to provide audible signals to and receive audible signals from the user. For example, in addition to being coupled to the audio transducer 625, the audio interface 674 may also be coupled to a microphone to receive audible input, such as to facilitate a telephone conversation. In accordance with embodiments of the present disclosure, the microphone may also serve as an audio sensor to facilitate control of notifications, as will be described below. The system 602 may further include a video interface 676 that enables an operation of an on-board camera 630 to record still images, video stream, and the like.

A mobile computing device 600 implementing the system 602 may have additional features or functionality. For example, the mobile computing device 600 may also include additional data storage devices (removable and/or non-removable) such as, magnetic disks, optical disks, or tape. Such additional storage is illustrated in FIG. 6B by the non-volatile storage area 668.

Data/information generated or captured by the mobile computing device 600 and stored via the system 602 may be stored locally on the mobile computing device 600, as described above, or the data may be stored on any number of storage media that may be accessed by the device via the radio interface layer 672 or via a wired connection between the mobile computing device 600 and a separate computing device associated with the mobile computing device 600, for example, a server computer in a distributed computing network, such as the Internet. As should be appreciated such data/information may be accessed via the mobile computing device 600 via the radio interface layer 672 or via a distributed computing network. Similarly, such data/information may be readily transferred between computing devices for storage and use according to well-known data/information transfer and storage means, including electronic mail and collaborative data/information sharing systems.

FIG. 7 illustrates one aspect of the architecture of a system for processing data received at a computing system from a remote source, such as a personal computer 704, tablet computing device 706, or mobile computing device 708, as described above. Content displayed at server device 702 may be stored in different communication channels or other storage types. For example, various documents may be stored using a directory service 722, a web portal 724, a mailbox service 726, an instant messaging store 728, or a social networking site 730.

One or more ML models or applications 720 may be employed by a client that communicates with server device 702, and/or ML Models/Applications 721 (e.g., performing aspects described herein) may be employed by server device 702. The server device 702 may provide data to and from a client computing device such as a personal computer 704, a tablet computing device 706 and/or a mobile computing device 708 (e.g., a smart phone) through a network 715. By way of example, the computer system described above may be embodied in a personal computer 704, a tablet computing device 706 and/or a mobile computing device 708 (e.g., a smart phone). Any of these embodiments of the computing devices may obtain content from the store 716, in addition to receiving graphical data useable to be either pre-processed at a graphic-originating system, or post-processed at a receiving computing system.

While the present disclosure has been described as having an exemplary design, the presently disclosed examples can be further modified within the spirit and scope of this disclosure. This application is therefore intended to cover any variations, uses, or adaptations of the aspects disclosed herein using its general principles. Further, this application is intended to cover such departures from the present disclosure as come within known or customary practices in the art to which this disclosure pertains. Some governing principles of interpreting this disclosure are provided here below for reference.

The connecting lines shown in the various figures contained herein are intended to represent exemplary functional relationships and/or physical couplings between the various elements. It should be noted that many alternative or additional functional relationships or physical connections can be present in a practical system. However, the benefits, advantages, solutions to problems, and any elements that can cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone can be present in an embodiment, B alone can be present in an embodiment, C alone can be present in an embodiment, or that any combination of the elements A, B or C can be present in a single embodiment; for example, A and B, A and C, B and C, or A and B and C.

In the detailed description herein, references to “one embodiment,” “an embodiment,” “an example embodiment,” etc., indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method operation in the present disclosure is intended to be dedicated to the public regardless of whether the element, component, or method operation is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus

What is claimed is:

1. A method of mitigating a stress level of a user, the method comprising:
collecting potential stress indicator data from the user interacting with a computing device, the potential stress indicator data comprising one or more of environmental data and contextual data associated with the user;
estimating, based on the potential stress indicator data, the stress level of the user;
performing an evaluation of whether to mitigate, via one or more stress mitigation interventions, the stress level of the user; and
presenting the one or more stress mitigation interventions to the user via a graphical user interface (GUI) when the evaluation indicates that the stress level should be mitigated.

2. The method of claim 1, wherein the computing device comprises at least one input device with which to perceive the potential stress indicator data and a display with which to present the one or more stress mitigation interventions.

3. The method of claim 2, wherein the computing device is a personal computer, and wherein the potential stress indicator data includes at least one of pointer activity, mouse activity, keyboard activity, and personal information manager activity.

4. The method of claim 1, wherein the environmental data comprises data that is indicative of one or more of physiological activity and behavioral activity of the user, and wherein the contextual data comprises data that is indicative of one or more of an amount of user interaction with the computing device and an amount of user activity away from the computing device.

5. The method of claim 1, wherein the method is performed via a machine learning stress mitigation architecture comprising a stress indicator model that is configured to identify actual stress indicator data in the collected potential stress indicator data and a stress estimator model that is configured to estimate the stress level of the user such that estimating, based on the potential stress indicator data, the stress level of the user comprises:
identifying, via the stress indicator model, actual stress indicator data from the collected potential stress indicator data; and
aggregating, via the stress estimator model, the actual stress indicator data into an estimated stress level of the user.

6. The method of claim 5, wherein the machine learning stress mitigation architecture further comprises a stress predictor model that is configured to predict a future stress level of the user based on the potential stress indicator data and wherein estimating, based on the potential stress indicator data, the stress level of the user further comprises:
predicting, via the stress predictor model, the future stress level of the user and
aggregating, via the stress estimator model, the future stress level of the user into the estimated stress level of the user.

7. The method of claim 6, wherein predicting the future stress level of the user includes determining a sentiment of stress indicator data from a personal information manager in communication with the computing device.

8. The method of claim 5, further comprising calibrating the stress estimator model via one or more user prompts that are configured to provide indication of an actual stress level of the user.

9. The method of claim 5, wherein the machine learning stress mitigation architecture includes an intervention efficacy model that is configured to determine one or more of an efficacy of the intervention on the stress level of the user and whether the user completes the intervention.

10. The method of claim 9, wherein the machine learning stress mitigation architecture includes a stress intervention generator model that is configured to vary at least one of a complexity and a type of the one or more stress mitigation interventions corresponding to at least one of the stress level and the efficacy of the one or more stress mitigation interventions.

11. The method of claim 1, wherein the GUI comprises a workspace with which to display applications running on the computing device, the GUI being configured to perform at least one of modifying the workspace to include a wellness widget, providing the one or more stress mitigating interventions to the user, and displaying an estimated stress of the user over time.

12. A method of estimating a stress level of a user interacting with a computing device, the method comprising:
selecting a baseline stress level of the user;
collecting potential stress indicator data from the user interacting with the computing device, the potential stress indicator data comprising one or more of environmental data and contextual data associated with the user;
estimating, based on the potential stress indicator data, the stress level of the user; and
returning the stress level to a graphical user interface (GUI) the computer device.

13. The method of claim 12, wherein estimating, based on the potential stress indicator data, the stress level of the user includes:
processing the potential stress indicator data to identify actual stress indicator data;
aggregating the actual stress indicator data to form the stress level of the user; and
comparing the stress level of the user to the baseline stress level of the user.

14. The method of claim 12, wherein estimating, based on the potential stress indicator data, the stress level of the user is performed via a stress estimator model, and wherein the method further comprises calibrating the stress estimator model via one or more user prompts that are configured to provide indication of an actual stress level of the user.

15. The method of claim 12, wherein estimating, based on the potential stress indicator data, the stress level of the user comprises at least one of determining a current stress level of the user and predicting a future stress level of the user.

16. The method of claim 15, wherein predicting the future stress level of the user includes determining a sentiment of stress indicator data from a personal information manager in communication with the system of computing devices.

17. A graphical user interface (GUI) for mitigating a stress level of a user, the GUI comprising a workspace with which to display applications running on a computing device, the GUI being configured to:
receive an indication that mitigation of the stress level of the user is recommended upon estimation of the stress level of the user based on potential stress indicator data from the user interacting with the system of computing devices associated with the user, the potential stress indicator data comprising one or more of environmental data and contextual data associated with the user; and
modify the workspace to include a wellness widget that is configured to provide a stress mitigating intervention to the user.

18. The GUI of claim 17, wherein modifying the workspace to include the wellness widget that is configured to provide the stress mitigating intervention to the user comprises modifying the workspace to include a first workspace for displaying the applications and a second workspace for displaying the wellness widget.

19. The GUI of claim 18, wherein the first and second workspaces are positioned side by side on the display, and wherein the first workspace is larger than the second workspace such that the wellness widget is defined as a sidebar on the workspace.

20. The GUI of claim 17, wherein the wellness widget is incorporated into an application displayed on the GUI and provides an indication of the stress level of the user over time.
